(19) United States
(12) Patent Application Publication
(10) Pub. No.: US 2006/0127478 A1
(43) Pub. Date: Jun. 15, 2006

(54) Oral dosage formulation

(76) Inventors: Horst G. Zerbe, QUEBEC (CA); Pompilia Szabo, Greenfield Park (CA)

Correspondence Address: GOUDREAU GAGE DUBUC, 800 PLACE VICTORIA, SUITE 3400, MONTREAL, QUEBEC, H4Z 1E9, CA

(21) Appl. No.: 10/542,983

(22) PCT Filed: Feb. 12, 2004
(86) PCT No.: PCT/CA04/00073

§ 371 Date: Jul. 21, 2005

Related U.S. Application Data
(60) Provisional application No. 60/441,156, filed on Jan. 21, 2003.

(51) Int. Cl. A61K 9/24

(52) U.S. Cl. 424/472

(57) ABSTRACT

A multi-layer oral dosage form, preferably a tablet, comprising a matrix core comprising a therapeutically effective amount of a first drug (NSAID), wherein the matrix core allows sustained release of the first drug; a first layer, which is in contact with the matrix core, comprising a first portion of a pharmaceutically effective amount of a second drug (H₂-blocker antagonist), wherein the first layer allows sustained release of the second drug; and a second layer, which is in contact with said matrix core, comprising a second portion of the second drug, wherein the second layer allows immediate release of the second drug. Methods for preparing the multi-layer dosage form are also disclosed.

### FIELD OF THE INVENTION

The present invention relates to oral dosage formulations. More particularly, the present invention relates oral dosage formulations comprising a non-steroidal anti-inflammatory drug (N SAID) and an H₂-receptor antagonist.

### BACKGROUND OF THE INVENTION

In recent years an increased interest in multi-layered tablets as controlled-release systems has been observed. Multi-layered tablets have some obvious advantages over conventional tablets, and are commonly used to avoid chemical incompatibilities between formulation components. These chemically incompatible formulation components, often biologically active ingredients (drugs), can be incorporated into one tablet by physically separating them into distinct layers. In the context of drug delivery systems, multi-layered tablets allow for the modification of release profiles, by combining layers with different release profiles, i.e. by combining slow-release with immediate-release layers.

Conte et al. (1) have proposed a controlled-release tablet called Geomatrix®, which is based on the multi-layered tablet concept. Functionally, the product represents a swellable matrix. The swelling of the drug-containing layer causes an increase of the surface area and therefore an increase in the amount of drug released per unit of time. Meanwhile, the outer cover layers control the diffusion of the drug from the drug containing layer. Other examples of products involving the multiple-layered tablet concept were published by Qiu et al. (2), Yang et al. (3), Abraham et al. (4), Nangia et al. (5), and Chidambaram et al. (6).

Complex multi-layered tablets are tablets having differently shaped layers. The shape of the outer layers depends on the shape of the tablet core (Zerbe and Krumme (7)). The concept of complex multi-layered tablets to achieve zero-order release from matrix-based systems, was first introduced by Cremer (U.S. Pat. No. 5,853,760) and by Cremer and Asmussen (8).

NSAIDs comprise a class of drugs having long been recognized as being of high therapeutic value in the treatment of inflammatory conditions. Despite their therapeutic benefits, the use of NSAIDs is frequently limited by an increased risk of gastrointestinal side-effects such as peptic ulceration and dyspeptic symptoms.

Attempts at modifying the NSAID structure in order to prevent such side-effects have been moderately successful at best. A more promising alternative to the problem of NSAID associated gastrointestinal side-effects, more particularly in patients with a need for continuous NSAID treatment, is to combine the NSAID with an anti-ulcer drug such as for example prostaglandin analogues, H₂-receptor antagonists such as for example omeprazole or sucralefate, or proton pump inhibitors. Yet another suggested alternative involves the administration of NSAIDs following the ingestion of food or milk.

The NSAID sodium diclofenac has been used for decades for the symptomatic treatment of osteoarthritis and rheumatoid arthritis. Famotidine, an H₂-receptor antagonist, has proven to be useful for the treatment of gastric and duodenal ulcers as well as for the relief of heartburn. Famotidine has also been shown to reduce the frequency of gastric and duodenal ulcers associated with non-selective NSAIDs such as diclofenac, ibuprofen, naproxen, and ketoprofen (Taha et al., New England Journal of Medicine, 1996; 334:1435-1437).

The frequency of gastric and duodenal ulcers associated with COX-2 inhibitors and non-selective NSAIDs in patients suffering from osteoarthritis and rheumatoid arthritis, as well as in a subset of these patients additionally taking low dosages of aspirin, has also been investigated. Commercially available COX-2 inhibitors such as Celebrex®, Vioxx® and Bextra®, have been shown to produce a lower frequency of gastroduodenal ulcers than non-selective NSAIDs. However, low dosages of aspirin administered with COX-2 inhibitors substantially increase the frequency of upper GI ulceration. This seems to indicate that COX-2 inhibitors do not offer sufficient protection against ulcers induced by low-dosages of aspirin, which in turn has important implications since a large portion of patients suffering from osteoarthritis and rheumatoid arthritis also ingest low dosages of aspirin.

Gimet et al. (U.S. Pat. No. 5,601,843) teach pharmaceutical compositions, more specifically a core/mantle tablet, comprising a core consisting of an NSAID which is either diclofenac or piroxicam, and a coating incorporating a prostaglandin such as misoprostol. Misoprostol, even though effectively preventing NSAID-induced gastroduodenal ulceration, is associated with a high incidence of adverse effects such as abdominal pain, diarrhea, nausea and flatulence.

Ouali et al. (U.S. Pat. No. 6,287,600) disclose pharmaceutical compositions for oral administration consisting of a bi-layer tablet comprising an NSAID and a prostaglandin, wherein the NSAID is enterically coated.

Woolfe at al. (U.S. Pat. No. 6,387,410) teach oral pharmaceutical compositions, more specifically multi-layer tablets comprising a mixture of a delayed release formulation of an NSAID and a mixture comprising a prostaglandin, wherein the NSAID formulation is in the form of coated beads or granules providing programmed release according to the position in the gastrointestinal tract.

Saslawski et al., (U.S. Pat. No. 6,372,255) teach multi-layer tablets for the instant and then prolonged release of active substances. The tablets comprise a first layer containing an active substance in the form of a granule which disintegrates immediately upon contact with an aqueous medium such as a physiological medium, and a second layer composed of an inert matrix wherein is dispersed a second active substance, and wherein the matrix allows for the prolonged release of the second active ingredient.

Depui et al. (U.S. Pat. No. 6,365,184) disclose an oral pharmaceutical dosage form comprising an NSAID (diclofenac) and an acid susceptible proton pump inhibitor (omeprazole). The proton pump inhibitor is generally in the form of an enterically coated pellet capable of compression into tablets together with the NSAID. The enteric coating layer has mechanical properties such that the acid resistance of the enterically coated pellets is not significantly affected by the compression of the pellets with the other components during tableting.

There thus remains a need to develop an improved oral dosage form comprising an extended-release NSAID and an H₂-receptor antagonist for the treatment of osteoarthritis in patients at an elevated risk for developing gastrointestinal side effects.

The present invention seeks to meet these and other needs.

The present invention refers to a number of documents, the content of which is herein incorporated by reference in their entirety.

### SUMMARY OF THE INVENTION

The present invention relates to a novel oral dosage form, preferably a tablet, more preferably a fixed-dose multi-layer tablet comprising two or more drug combinations, as well as to methods of making the multi-layer tablet. Most preferably, the present invention relates to fixed-dose combination tablets comprising an NSAID and an H₂-receptor antagonist. Still most preferably, the present invention relates to an improved fixed-dose multi-layer tablet comprising an extended-release NSAID as well as an H₂-receptor antagonist, useful for the treatment of osteoarthritis in patients who are at an elevated risk for developing gastrointestinal side effects, more specifically NSAID-induced gastric and duodenal ulcers. Yet even more preferably, the present invention relates to an improved fixed-dose multi-layer tablet comprising an extended-release NSAID as well as an H₂-receptor antagonist, useful for the treatment of osteoarthritis in patients who are at an elevated risk for developing gastrointestinal side effects, more specifically NSAID-induced gastric and duodenal ulcers, and who are also taking low doses of aspirin for the prevention of myocardial infarction.

In a preferred embodiment, the present invention relates to a multi-layer oral dosage form comprising a matrix core comprising a therapeutically effective amount of a first drug, wherein the matrix core allows sustained release of the first drug; a first layer, which is in contact with the matrix core, comprising a first portion of a pharmaceutically effective amount of a second drug and optionally an additional amount of the first drug, wherein the first layer allows sustained release of the first and second drug; and a second layer, which is in contact with the matrix core, comprising a second portion of the second drug, wherein the second layer allows immediate release of the second drug.

The present invention may also relate to a multi-layer oral dosage form comprising a matrix core comprising a therapeutically effective amount of a first drug, wherein the matrix core allows sustained release of the first drug; a first layer, which is in contact with the matrix core, comprising a first portion of a pharmaceutically effective amount of a second drug, wherein the first layer allows sustained release of the second drug; and a second layer, which is in contact with the matrix core, comprising a second portion of the second drug, wherein the second layer allows immediate release of the second drug.

The present invention may also relates to a method for preparing a multi-layer oral dosage form comprising:
(a) preparing a sustained release matrix core comprising a therapeutically effective amount of a first drug or pharmaceutically acceptable salts thereof;
(b) preparing a sustained release blend comprising a first portion of a pharmaceutically effective amount of a second drug or pharmaceutically acceptable salts thereof;
(c) preparing an immediate release blend comprising a second portion of the second drug or pharmaceutically acceptable salts thereof; and
(d) combining, by compressing, the matrix core of step (a), the sustained release blend of step (b) and the immediate release blend of step (c).

The present invention may also relate to new oral pharmaceutical compositions for use in the treatment and prophylaxis of gastrointestinal disorders associated with the use of Non Steroidal Anti-Inflammatory Drugs (NSAIDs).

The present invention may also relate to pharmaceutical compositions comprising a combination of a non-steroidal anti-inflammatory drug and an H₂-receptor antagonist as well as to methods of preparing such compositions.

In related embodiments, the present invention relates to an improved fixed-dose pharmaceutical formulation comprising an extended-release NSAID and an H₂-receptor antagonist, wherein a first portion of the H₂-receptor antagonist is released following an immediate release profile and wherein a second portion is released following an extended release profile.

The present invention may also relate to a method for reducing the undesirable gastrointestinal side effects associated with the oral administration of NSAIDs, comprising administering a fixed-dose multi-layer tablet containing an NSAID and an H₂-receptor antagonist to a patient in need thereof.

In a further preferred embodiment, the present invention relates to a fixed-dose multi-layer tablet comprising an extended release NSAID and an H₂-receptor antagonist, wherein the NSAID is diclofenac and wherein the H₂-receptor antagonist is famotidine.

In another preferred embodiment, the present invention relates to a method for treating or preventing osteoarthritis in patients at an elevated risk for developing gastrointestinal side effects, more specifically NSAID induced gastric and duodenal ulcers, comprising administering an effective amount of a fixed-dose multi-layer tablet comprising an extended release NSAID and an H₂-receptor antagonist, wherein the NSAID is diclofenac and wherein the H₂-receptor antagonist is famotidine.

In yet another preferred embodiment, the present invention relates to a method for treating or preventing osteoarthritis in patients at an elevated risk for developing gastrointestinal side effects, more specifically NSAID induced gastric and duodenal ulcers, and who are also taking low doses of aspirin for the prevention of myocardial infarction, comprising administering an effective amount of a fixed-dose multi-layer tablet comprising an extended release NSAID and an H₂-receptor antagonist, wherein the NSAID is diclofenac and wherein the H₂-receptor antagonist is famotidine.

Further scope and applicability will become apparent from the detailed description given hereinafter. It should be understood however, that this detailed description, while indicating preferred embodiments of the invention, is given by way of illustration only, since various changes and modifications within the spirit and scope of the invention will become apparent to those skilled in the art.

### BRIEF DESCRIPTION OF THE DRAWINGS

FIG. 1 shows a multi-layered dosage form comprising: an immediate release layer (IR) comprising X mg of drug A, a sustained release layer (SR) comprising Y mg of drug A, as well as a sustained release core comprising Z mg of drug B;

FIG. 2 shows a combined in-vitro dissolution profile of an immediate release (IR) and a sustained release (SR) layer containing famotidine (drug A), obtained in SGF (Simulated Gastric Fluid) at 100 rpm and 37° C.;

FIG. 3 shows an in vitro dissolution profile for a sustained release (SR) core comprising diclofenac (drug B), obtained in SIF (Simulated Intestinal Fluid);

FIG. 4 shows in vitro dissolution profiles obtained simultaneously from a multi-layer tablet comprising famotidine (drug A), subdivided into immediate and sustained release layers, and diclofenac (drug B), present in a sustained release core, obtained in SIF (Simulated Intestinal Fluid) at 100 rpm; and

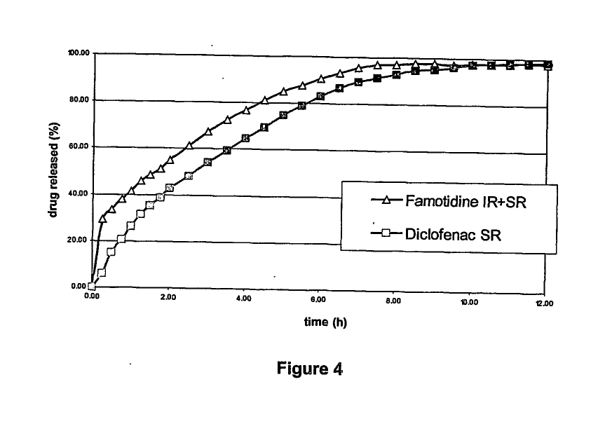

FIG. 5 shows an in vitro dissolution profile for a sustained release (SR) core comprising aspirin as well as for a sustained release (SR) core comprising aspirin and which is integrated in a multi-layer tablet obtained in SIF (Simulated Intestinal Fluid) at 100 rpm.

### DETAILED DESCRIPTION OF THE INVENTION

The terms “active agent”, “active ingredient”, “drug” and “pharmaceutically active agent” are used interchangeably herein, and are meant to refer to a compound which, when administered to a human or an animal induces, a pharmacological effect.

As used herein, the term “effective amount” or “therapeutically effective amount” is well known in the art. It is meant to describe a non-toxic but sufficient amount of the agent capable of providing a desired therapeutic effect. An appropriate “effective amount” in any individual case may be determined by one of ordinary skill in the art using only routine experimentation.

As used herein, the term “erosion” is given the general meaning as commonly accepted in the pharmaceutical arts. The term “erosion” is generally accepted in the pharmaceutical arts as being a process in which solid masses are cleared away.

As used herein, the term “prodrug” refers to an inactive form of a drug, that exerts its effects after metabolic processes within the body convert it to a usable or active form. The usable or active form is generally the active form of the drug prior to conversion into a prodrug.

As used herein, the term “oral dosage formulation” refers to a pharmaceutical composition comprising a therapeutically effective amount of the active agent optionally in addition with pharmaceutically acceptable excipients, which may be orally administered. For oral administration, the formulation may take the form of tablets, caplets, lozenges or capsules, formulated in a conventional manner.

The pathogenesis of NSAID-induced gastroduodenal mucosal injury encompasses topical injury as well as systemic mechanisms. Topical mucosal injury is believed to be mediated by the inherent acidic properties of aspirin as well as many other NSAIDs. Systemic effects are thought to be largely the result of the inhibition of endogenous prostaglandin synthesis.

Pharmaceutical formulations wherein an NSAID such as for example diclofenac is combined with an H₂-receptor antagonist, such as for example famotidine, are useful in helping and/or preventing NSAID-induced ulcers in patients suffering from osteoarthritis and rheumatoid arthritis, in addition to helping to prevent aspirin induced ulceration.

In a broad sense, the present invention relates to novel oral pharmaceutical compositions comprising an NSAID and an H₂-receptor antagonist, capable of addressing both topical or systemic mechanisms of NSAID-induced gastroduodenal mucosal injury.

Preferably, the present invention is embodied in an improved fixed-dose combination tablet comprising an sustained release NSAID and an H₂-receptor antagonist, wherein a first portion of the H₂-receptor antagonist is released in the gastroduodenal lumen following an immediate release profile, followed by the concomitant release of a second portion of the H₂-receptor antagonist and the NSAID. The second portion of the H₂-receptor antagonist and the NSAID are released following sustained (extended) release profiles. The immediately released portion of the H₂-receptor antagonist addresses any possible topical ulcerogenic effects, whereas the sustained portion addresses any systemic ulcerogenic effects of the NSAID.

In one particular embodiment, the present invention relates to an improved fixed-dose multi-layer tablet comprising a sustained release NSAID and an H₂-receptor antagonist, wherein the NSAID is diclofenac and wherein the H₂-receptor antagonist is famotidine.

Other non-limiting examples of NSAIDs that can be incorporated into the multi-layer tablets as defined herein comprise ibuprofen, naproxen, flurbiprofen, alminoprofen, and tiaprofenic acid. Other non-limiting examples of H₂-blocker antagonists that can be incorporated into the multi-layer tablets as defined herein comprise ranitidine, nizatidine, cimetidine, and roxatidine.

The multi-layer tablet is preferably prepared by first producing the NSAID containing core (in the form of a layer structure), followed by at least partially coating it with an erodable layer comprising a first portion of the H₂-receptor antagonist, providing for a sustained release layer of the antagonist. An immediate release layer comprising a second portion of the H₂-receptor antagonist is then applied.

Diclofenac is an NSAID having acidic properties. The design of sustained release formulations comprising one or more drugs having acidic properties represents an important technological challenge. The low pH environment commonly encountered in the stomach suppresses the ionization of acidic drugs, thus considerably reducing the solubility of these drugs in gastric juices. A pH increase, as is observed in the intestines, results in a solubility increase and a faster release rate. The fixed-dose multi-layer pharmaceutical formulations of the present invention ensure essentially constant blood plasma levels of acidic drugs, such as for example diclofenac, throughout the digestive tract.

Famotidine's primary pharmacological function is the inhibition of gastric secretion. Famotidine was shown to inhibit basal and nocturnal gastric secretion, as well as food and pentagastrin stimulated secretion, one hour following oral administration. The maximum effect is dose dependent, and was observed within one to three hours following oral administration. Doses of 20 mg and 40 mg effectively inhibit gastric secretion over periods ranging from 10 to 12 hours. The nocturnal intra-gastric pH was raised to mean values of 5.0 and 6.4 following nocturnal doses of 20 mg and 40 mg respectively. The basal daytime inter-digestive pH, at three and eight hours following the administration after breakfast of 20 or 40 mg of famotidine, was raised to about 5 (Physician's Desk Reference, 2001).

A fixed-dose multi-layer tablet as described herein and comprising a combination of an NSAID and an H₂-receptor antagonist, wherein the NSAID is formulated such as to be released following a sustained release profile, and wherein the H₂-receptor antagonist is formulated such that a first portion is released following an immediate release profile and wherein a second portion is released following a sustained release profile, provides for better patient compliance and increased efficiency of the NSAID. The fixed dose pharmaceutical compositions of the present invention are so formulated that a first portion of the H₂-receptor antagonist is released, followed by the concomitant release of the NSAID and a second portion of the H₂-receptor antagonist. The suppression of gastric secretion by the H₂-receptor antagonist significantly reduces the rate of occurrence of ulceration, in addition to increasing the intra-gastric pH which favorably effects the solubility and absorbance of the NSAID.

Tablet Design

The pharmaceutical compositions of the present invention are multi-layered solid fixed-dosage forms, more preferably multi-layered fixed-dose combination tablets. The compositions can be administered once-daily or twicedaily, depending on the dosage of the active components. Both dosage forms provide for sufficient plasma levels for the treatment of osteoarthritis, while at the same time preventing gastrointestinal side effects, more specifically the formation of NSAID induced gastric and duodenal ulcers. This is of particular benefit to patients also taking low dosages of aspirin as a preventive measure against myocardial infarction. The H₂-receptor antagonist is released following two distinct release profiles; a first portion being released following an immediate release profile and a second portion being released following an extended release profile.

Several systems capable of providing for the controlled release of pharmaceutical agents, such as diffusion systems (including reservoir devices and inert polymeric matrices), erodable systems (based on the inherent dissolution of the drug itself), and osmotic systems (drug containing core coated with a semi-permeable membrane having a small orifice) have been investigated and published (9-14).

The controlled release of a drug from a pharmaceutical dosage form can also be achieved by more than one mechanism. For example, for the same pharmaceutical dosage form, the drug release can occur for example by simultaneous swelling and diffusion, simultaneous diffusion and erosion, and simultaneous swelling, diffusion and erosion.

In the case of matrix systems, the rate of drug release is largely dependant on the properties of the composition used to make the matrix, on the physical properties and concentration of the active as well as on the geometry of the matrix. Tablet diameter and surface area of the tablet are additional factors influencing the rate of drug release.

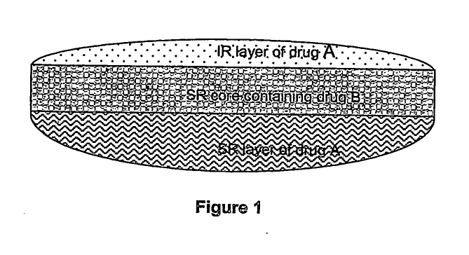

The fixed-dose multi-layer tablets of the present invention are useful for the treatment of osteoarthritis in patients who are at an elevated risk for developing gastrointestinal side effects, more specifically NSAID-induced gastric and duodenal ulcers. The fixed-dose combination tablets include a pharmaceutical formulation comprising at least two active ingredients, more preferably two active ingredients (FIG. 1). The first active ingredient is an H₂-blocker antagonist, divided into a first portion formulated as an immediate release layer, and a second portion formulated as a sustained release layer.

The pharmaceutical formulation providing the immediate release layer is conceived to rapidly disintegrate, and will preferably contain from about 5 to about 25% of the H₂-receptor antagonist. Moreover, the pharmaceutical formulation providing the immediate release layer is comprised of a dry mixture of the drug (H₂-blocker antagonist) and pharmaceutically acceptable excipients such as for example polysaccharides and their derivatives, cross-linked polymers, soluble salts, disintegrants and other excipients well known by a person skilled in the art. Additives such as colorants, fillers, anti-tacking and anti-static agents may also be incorporated into the formulation. Non-limiting examples of such additives are magnesium stearate and talc.

The pharmaceutical formulation providing the sustained release layer will preferably contain from about 75 to about 95% of the H₂-receptor antagonist. The H₂-receptor antagonist present in the sustained release layer may be directly mixed with pharmaceutically acceptable excipients or it may be first coated with hydrophilic or hydrophobic agents, which are specifically chosen to regulate the rate of release of the antagonist. The sustained release layer may be further comprised of polymeric materials, which are slowly water-soluble and/or slowly gel-forming when exposed to an aqueous medium. Non-limiting examples of such polymeric materials are cellulose derivatives and modified starches. The sustained release layer is then applied to an NSAID containing core. The thickness of the sustained release layer can be varied, depending on the specific requirements.

The sustained release layer has a direct impact on the rate of release of the NSAID from the core, and provides for a sustained release of the H₂ receptor antagonist. In one particular embodiment, an erodable mass of solids can be incorporated into the sustained release layer in order to adjust the release of the NSAID from the core. It is understood that an increased amount of erodable mass incorporated into the sustained release layer will result in an increase in the amount of NSAID released from the core. In another embodiment, the sustained release layer is formulated to provide an essentially stable layer from which the H₂ receptor antagonist diffuses at a sustained rate, while simultaneously providing for a sustained release of the NSAID from the core.

Matrix-forming excipients are commonly used to ensure a sustained release of pharmaceutically active agents. Such materials form a hydrophilic/hydrophobic matrix, providing for the sustained release of the active following both diffusion and erosion mechanisms. Hydrophilic drugs are predominantly released from the matrix following diffusion mechanisms. Surface area fluctuations play an important role in those cases where erosion is the leading factor in controlling the rate of drug release.

An erodable mass is commonly generated by specific grades of polymers and combinations thereof optionally in association with various fillers. Non limiting examples of polymers include polysaccharides, polylactides, polyglycolides, polyethylenes and polypropylenes, metacrylates, polyvinylchlorides and polyvinyl chlorides and polyvinyl pyrolidones.

As mentioned previously, the fixed-dose combination tablets include a pharmaceutical formulation comprising at least two active ingredients, more preferably two active ingredients (FIG. 1), the H₂-blocker antagonist being the first active ingredient. The second active ingredient is an NSAID. The NSAID is formulated as a separate sustained release layer, more specifically the core of the multi-layer fixed-dose combination tablet as described herein. The sustained release core will preferably contain from about 50 to about 100% of the recommended daily dose of the NSAID. The NSAID containing sustained release core is prepared in accordance with known techniques in the art. The core composition comprises an easily flowable homogeneous mixture that is compressed under a pressure ranging from about 3 to about 15 kN. The sustained release core represents a non-erodable structure from which the NSAID diffuses at a sustained rate into the surrounding media.

The NSAID comprising core formulation is commonly generated by combinations of specific polymers, optionally in association with adjuvants. Non-limiting examples of polymers that can be used in the NSAID comprising core formulation are Insoluble cellulose-based materials, polyvinyl acetates, polyvinyl alcohols, polyethylene oxides, metacrylates and non-crosslinked polyvinylpyrolidone. Non-limiting examples of adjuvants are sucrose, lactose, colloidal silica and magnesium stearate. The ratio of polymer to active agent (NSAID) in the core formulation varies with the type of active ingredient.

Multi-layer tablets possess numerous advantages in comparison to conventional dosage forms. Chemically incompatible components can be incorporated into a multi-layer tablet by integrating them into separate layers. Moreover, a different active ingredient can be incorporated into one or more of the distinct layers of the multi-layer tablet, thereby offering the possibility of designing each layer so as to obtain a desired release profile for each active ingredient, thus maximizing both their individual and combined therapeutic effect.

Tablets may be designed to have pulsatile, immediate onset, delayed onset or any other suitable predetermined release profile. The different layers of the fixed-dose multi-layer tablet as described herein may comprise different active agents, different amounts of active agent and/or different forms of active agent. Moreover, the various layers of the fixed-dose multi-layer tablet as described herein, may comprise different amounts of one or more polymers as well as different kinds of additional pharmaceutical excipients, thus providing for additional control of the release of the active agents from the tablet.

As the fixed-dose multi-layer tablet passes through the digestive tract, it releases varying amounts of active or active agents depending on its location in the digestive tract (i.e. stomach, versus small intestine versus colon). A predetermined release scheme can thus be rationally designed for the active or active agents comprised in the fixed-dose multi-layer tablet, based on the formulation of the different layers. It may be desirable that a first active agent be released in the upper digestive tract (e.g., stomach or small intestine) while a second active agent is released in the lower digestive tract. Alternatively, it may be desirable that a portion of a first active ingredient be released in the upper digestive tract (e.g., stomach or small intestine) while a second portion of the first active agent and the second active agent be released in the lower digestive tract.

The pharmaceutical compositions of the present invention comprise a combination of famotidine and diclofenac, wherein a first portion of famotidine is released in the upper digestive tract while a second portion is released in the lower digestive tract together with diclofenac. The first portion of released famotidine is essentially provided by the immediate release layer of the fixed-dose multi-layer tablet, whereas the second portion is essentially provided by the sustained release layer of the multi-layer tablet. However, it is to be noted that small amounts of famotidine may be released from the sustained release layer as part of the first portion.

Active components having different water solubilities, requiring different dosages, and having different absorption profiles, can be formulated into a multi-layered tablet. A multi-layer (two or more layers) combination tablet as described herein allows for the controlled release of the active agent(s). Furthermore, the multi-layer combination tablet as described herein, provides for the combination of famotidine and diclofenac in such a way that the bioavailability is essentially similar to that of a separate administration of each active. A suitable ratio of the two active ingredients (diclofenac and famotidine) into a single dosage form, provides many important advantages from a therapeutic perspective.

Diclofenac is released from the core by diffusion, displaying a Fickian release profile. However, when the core is covered with erodable layers, the controlled erosion of these outer layers results in a steady increase of the surface area available for the release of the drug, thus providing a linear drug release.

Diclofenac is present in the multi-layer fixed-dose combination tablet as described herein, in a therapeutically effective amount. Preferably, the combination tablet is administered in unit dosage form. Preferably, the multi-layer fixed-dose combination tablet as described herein will comprise from about 50 to about 150 mg of diclofenac, and more preferably about 75 mg.

The active agents of the present composition, i.e., both the NSAID (diclofenac) and the H₂-blocker antagonist (famotidine) may be administered in the form of a pharmaceutically acceptable salt, ester, amide, prodrug or analog, or as a combination thereof. Salts, esters, amides, prodrugs and analogs of the active agents may be prepared using standard procedures known to those skilled in the art of synthetic organic chemistry (March, J., “Advanced Organic Chemistry: Reactions, Mechanisms, and Structure”, 4^{th} Edition (John Wiley & Sons, New York, 1992).

It is to be understood that the present invention is not to be limited to fixed dose combination tablets comprising an NSAID such as diclofenac, and an H₂-receptor antagonist such as famotidine. Other non-limiting examples comprise ibuprofen/famotidine; aspirin/famotidine; morphine/diclofenac; pioglitazone/metformin; ACE-I/statin; and ACE-I/β-blocker.

In a preferred embodiment of the present invention, the potential for gastric erosion is reduced by ensuring that a sufficient amount of famotidine is released before the release of diclofenac. The immediate release of famotidine helps raise the pH of the gastric fluid, which in turn aids in the dissolution of diclofenac.

### EXAMPLES

### Example 1

### Formulation of an Immediate Release Layer, Comprising Famotidine

The immediate release layer contains from about 5 to about 30% of the H₂ receptor antagonist famotidine, homogeneously mixed with a disintegrant, in a ratio ranging from about 1:10 to about 2:8.

A 100 mg layer containing 10% of the total amount of famotidine in addition to microcrystalline cellulose (Avicel PH 102 grade, Dow Chemical) was prepared. The bulk drug was sieved prior to use and dry-mixed with the polymer. The compression was performed in a Korch EK 0 tableting machine using a round die (diameter 10.0 mm).

### Example 2

### Formulation of a Sustained Release Layer, Comprising Famotidine

The sustained release layer containing famotidine possesses narrowly defined erosive properties and, at the same time, maintains good bonding to the core. The erosion rate of the core-covering layer has to be adjusted to match the intended release rate of famotidine, while providing the required continuous increase in exposed surface area for the release of diclofenac from the core, over the duration of time of the dosage.

An erodable layer can be typically manufactured by dry blending a mixture comprising from about 5 to about 40% of famotidine, from about 5% to about 50% of a hydrophilic polymer, from about 5% to about 50% of a hydrophobic polymer, and less then about 2% of a lubricant (magnesium stearate).

In a preferred embodiment, a portion of the famotidine is formulated as a sustained release layer. More preferably, the sustained release layer will comprise from about 15 to about 80% of the total amount of famotidine formulated in the multi-layer combination tablet. An erodable sustained release layer weighing about 150 mg, comprises about 30% hydroxypropylmethyl cellulose (Methocel K100), about 20% ethyl cellulose (Ethocel EC-20™), about 5-30% lactose, and about 1% of a lubricant. The mixture was compressed in a Korch EK 0 tableting machine, using a round die (diameter of 10.0 mm).

In another preferred embodiment, the erodable layer weighing about 1.00 mg comprises from about 15 to about 80% of the total amount of famotidine formulated in the multi-layer tablet, about 40% hydroxypropylmethyl cellulose (Methocel K100), from about 5 to about 15% ethyl cellulose (Aqualon N 100), from about 10 to about 30% lactose monohydrate, and about 1% of a lubricant. Compositions having enhanced compressibility and flow characteristics are obtained using dry/wet granulation.

The immediate release layer (Example 1) and the sustained release layer (Example 2), following respective pre-compression, were combined and compressed into a tablet following multi-layer technology using a rotary press. In one embodiment, the rotary press may contain the pre-compressed immediate release layer, to which is added the precompressed sustained release layer.

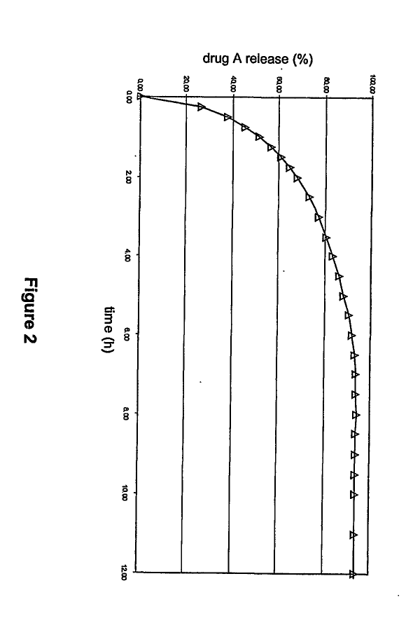

In vitro dissolution tests were conducted with tablets based on the formulations of Examples 1 and 2, using Apparatus 11 and the method detailed in USP 25. The stirrer paddle speed of the apparatus was 100 rpm, and the temperature of the medium was maintained at 37° C. The dissolution was observed at pH 1 (in simulated gastric fluid—SGF). Aliquot samples were assayed for famotidine by UV spectrophotometric measurements and the test results are shown in FIG. 2.

### Example 3

### Preparation of a SR Core Containing Diclofenac

The sustained release matrix comprising diclofenac is preferably provided as a non-erodable core made in accordance with the following steps:
a) intimately blending a pharmaceutically acceptable salt of diclofenac (from about 10 to about 40% by weight) with ethylcellulose (from about 5 to about 30% by weight; preferably EC-22, Aqualon) and a channeling agent, preferably lactose monohydrate (from about 25 to about 70% by weight) in a planetary or high shear mixer;
b) adding to the homogeneous blend from step (a), a solution of ethylcellulose (about 10% or less of ethylcellulose dissolved in ethanol) and monitoring the granulation process in order to obtain a uniform and complete distribution of the granulation liquid in the powder blend.

The release properties of the drug (diclofenac) from the core are dependent on the ratio of soluble to insoluble components, their particle sizes, the level of compaction, and the remaining porosity of the system. In addition to the physicochemical properties of the powder materials, the homogeneity of the blend and the distribution of the binders throughout the mix are essential. Consequently, the processing conditions selected for the granulation process determine the porosity of the granules and, eventually, the compression parameters of the final tablet. Throughout the process, the viscosity, the mixer speed and the chopper speed are parameters that are constantly monitored.
c) passing the composition through a 1.70 mm mesh;
d) drying the wet granules at about 50-60° C.;
e) size reducing the dried granules in a mill (preferably a Hammer mill) to obtain a granule size of less than 850 microns;
f) homogeneously blending the milled granules with a flowing agent such as silicone dioxide (less than about 4%) in a blender;
g) dry blending the mixture with a lubricant such as magnesium stearate (about or less than 2%); and
h) compressing the composition under a force ranging from about 3 kN to about 15 kN.

The non-erodable core could also be obtained by dry granulation followed by direct compression using blends comprising diclofenac (from about 25 to about 45% by weight), physical mixtures of polyvinyl acetate and polyvinyl pyrolidone (from about 20 to about 60% by weight), polyethylene oxides (from about 2 to about 10% by weight), silicon dioxide (from about 1 to about 3% by weight) and magnesium stearate (less then about 3% by weight).

SR formulations for a 200 mg core comprising diclofenac were prepared using various diclofenac/polymer ratios (physical mixtures of polyvinyl acetate and polyvinyl pyrrolidone) (i.e. 1:1, 1:1.5 and 1:2). A more preferred diclofenac/polymer ratio is 1:2. A dry-mixture of powders was passed through a 30 mesh screen and extra glidands and lubricants were added in a proportion of about 1% by weight (relative to the total core weight) for each excipient.

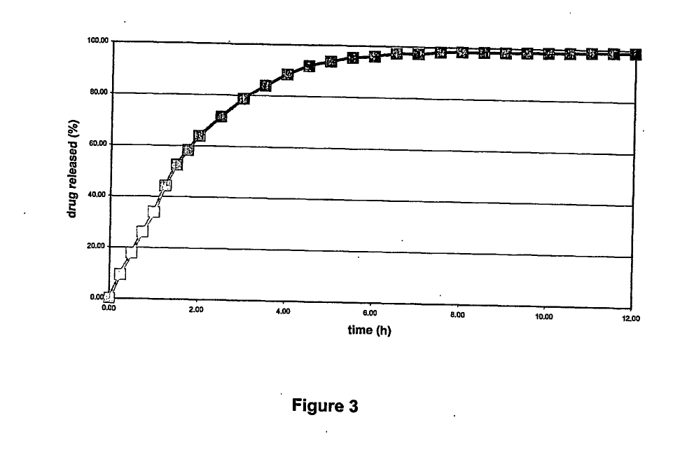

The mixture was compressed at a compression force of about 10 kN in a Korch EK 0 tableting machine having a round die (diameter of 9.8 mm). The influence of the compression force on the mechanical properties of the core, on the interlayer binding as well as on the in vitro dissolution profiles was studied. It was found that varying degrees of core hardness do not affect the dissolution of the drug in an aqueous medium. However, a very high compression force could induce weak interlayer binding. FIG. 3 illustrates a release profile of diclofenac (diclofenac/polymer ratio is 1:1) in SIF medium.

### Example 4

### Preparation of a SR Core Containing Aspirin (80 mg)

The sustained release matrix comprising aspirin is preferably provided as a non-erodable core made in accordance with the steps as previously described for diclofenac (Example 3).

The non-erodable core could also be obtained by direct compression using blends comprising aspirin (from about 25 to about 50% by weight), physical mixtures of polyvinyl acetate and polyvinyl pyrolidone (from about 20 to about 60% by weight), and magnesium stearate (less then about 2%).

SR formulations for a 270 mg core comprising aspirin were prepared using various drug aspirin/polymer ratios (physical mixtures of polyvinyl acetate and polyvinyl pyrrolidone) (i.e. 1:1, 1:1.5 and 1:2). A more preferred aspirin/polymer ratio is 1:2. A dry-mixture of granulated and regular powders was obtained and extra glidants and lubricants were added in a proportion of about 1% by weight (relative to the total core weight) for each excipient.

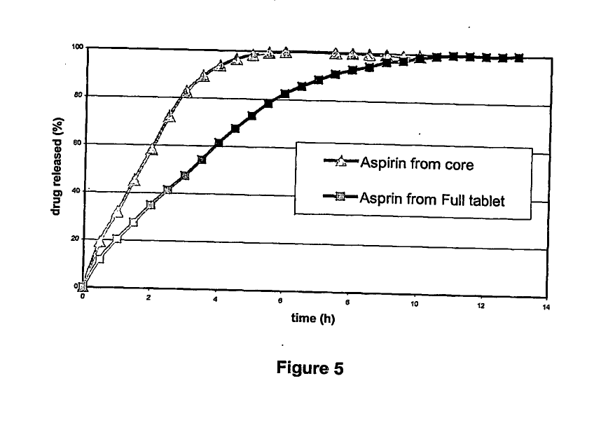

The mixture was compressed at a compression force of about 10 KN in a Korch EK 0 tableting machine having a round die (diameter of 7.0 mm.). The influence of the compression force on the mechanical properties of the core, on the interlayer binding as well as on the in vitro dissolution profiles was studied. It was found that varying degrees of core hardness do not affect the dissolution of the drug in an aqueous medium. FIG. 5 illustrates a release profile of aspirin from a SR matrix-core, as well as from a tablet comprising famotidine IR+SR layers in SIF medium.

### Example 5

### Manufacture of a Multi-Layer Fixed-Dose Combination Tablet

Using multi-layer technology, diclofenac (drug B) was compressed in a SR core. The core was then transferred into a rotary press containing either the IR or SR blend comprising famotidine (drug A). In one embodiment, the rotary press may contain the pre-compressed famotidine comprising IR layer, to which is added the diclofenac comprising core. Subsequent a first compression, the famotidine comprising SR layer is added followed by a final compression at a force of about 25 kN. This allows for a three-layer tablet to be independently processed using wet or dry granulated materials, as needed to enhance flow or compressibility.

### Example 6

### Manufacture of a Multi-Layer Tablet

Using multi-layer technology, aspirin (drug B) was compressed in a SR core The core was then transferred into a rotary press, containing either the IR or SR blend comprising famotidine (drug A). In one embodiment, the rotary press may contain the famotidine comprising IR layer, to which is added the diclofenac comprising core. Subsequent a first compression, the famotidine comprising SR layer is added followed by a final compression at a force of about 25 kN. This allows for a three-layer tablet to be independently processed using wet or dry granulated materials, as needed to enhance flow or compressibility.

### General Procedures

Dry granulation, fluidization, wet granulation, and extrusion are some of the methods commonly used for preparing the materials to be included in a solid dosage form.

Dry granulation procedures may be utilized where one of the components of the formulation, either the drug or the diluent, has insufficient cohesive or flow properties to be tabletted. The method includes mixing the ingredients, slugging the ingredients, dry screening, lubricating and finally compressing the ingredients.

An active agent can be pelletized or granulated using any suitable method known in the art. Pelletization or granulation is commonly defined as a size-enlargement process in which small particles are gathered into larger, permanent aggregates, in which the original particles can still be identified. Prior to granulation, a binder can be added to the active agent in order to improve the granulation process. Solvents and binders are typically added to a formulation to provide larger aggregates of granules. The temperature during granulation is generally not exceeding the melting point of any one of the components of the formulation. Typically, the mixture is granulated at a temperature ranging from about 35° C. to about 65° C. over a period ranging from about 10 to about 30 minutes. The granules are then typically air dried for a suitable duration of time (e.g. one or more hours). Preferably, the active agents are granulated using high shear mixer granulation or fluid-bed granulation. Both of these granulation processes provide enlarged granules or pellets, but differ in the apparatus used. In high shear mixing, blending and wet massing is accomplished by high mechanical agitation using an impeller and a chopper.

Fluidized bed granulation is a process in which granules are produced by spraying a binder solution onto a fluidized powder bed. The binder solution can be sprayed, for example, from a spray gun positioned in any suitable manner (e.g., top or bottom). The spray position and the rate of spraying may depend on the nature of the active agent and the binder used, and can be readily determined by those skilled in the art.

Optionally, granulated active agents can be milled. The mesh size of the screen can be selected depending on the size of the active agent granule or pellet desired. Typically, the mesh size can range from about mesh 20 to about mesh 100. The milling process aids in providing relatively uniform active agent granules.

Typically, the mean size of the active agent granule or pellet can range from about 50 μm to about 3 mm; preferably from about 100 μm to about 2 mm; or more preferably from about 300 μm to about 1 mm.

The bulk density or the tap density of the active agent granules or pellets ranges from about 0.1 g/ml to about 1.5 g/ml, preferably from about 0.3 g/ml to about 0.8 g/ml, or more preferably from about 0.4 g/ml to about 0.6 g/ml. The bulk density is measured based on the USP method.

Direct compression involves directly compressing the powdered material(s) to be included in the solid dosage form, without modifying the physical nature of the material itself.

### Compression into Tablets

Tableting can be accomplished using a tablet press. The tablet is formed by applying pressure on the lower and upper punches. Typical compression pressures range from about 6 kN to about 30 kN and will vary based on the desired size and hardness of the tablet. Preferably, the compression pressure is adjusted depending on the formulation characteristics and on the interlayer binding. Strong interlayer binding, more specifically between cover layers and the core matrix layer, is mandatory in order to ensure an erosion-controlled linear release of the drug from the core matrix. The physicochemical properties of the formulations are important factors influencing interlayer binding, as are the surface roughness and the hardness of the core. These properties and characteristics have a direct impact on the susceptibility to further compression The pre-compression force is therefore an essential parameter. If the compaction of the core granules exceeds a certain range, a tightly packed, “closed” core surface is formed. In such a tightly packed core, no penetration of particles of the cover layer into the core layer will occur during the main compression, which is essential for the formation of a strong bond between the two layers.

### Materials

One or more binders may be present in the pharmaceutical formulations in addition to, or in lieu of the fillers, in an amount ranging from about 0 to about 35%, and preferably from about 0.5 to about 30% by weight of the composition. Non-limiting examples of such binders, suitable for use herein, include polymeric materials (from natural or synthetic sources), sugars, salts, as well as wax binders such as carnauba wax, paraffin, spermaceti, or microcrystalline wax.

The polymeric material is a member selected from the group consisting of chitosan, modified starches, zein, maltodextrin, methylcellulose, ethylcellulose, hydroxyethylcellulose, hydroxypropylcellulose, hydroxypropylmethylcellulose, cellulose acetate, cellulose acetate butyrate, cellulose acetate propionate, cellulose acetate phthalate, hydroxypropylmethylcellulose phthalate, polyacrylic acid, metacrylate copolymers, polyvinyl acetate, polyvinylacetate phthalate, polyvinyl alcohol, polyethylene oxide, polyethylene glycol, polyvinyl pyrrolidone, polylactic acid, polyglycolic acid, polylactic/glycolic acid, polydimethyl silicone, polyhydroxyethyl metacrylate, polyethylene/vinyl acetate, polyethylene/vinyl alcohol, and mixtures thereof.

The pharmaceutical compositions as described herein are in the form of a tablet, and will include one or more tableting lubricants in an amount ranging from about 0.2 to about 8% and preferably from about 0.5 to about 2% by weight of the composition. Non-limiting examples of such lubricants are magnesium stearate, stearic acid, palmitic acid, calcium stearate, and the like. Other conventional ingredients, which may optionally be present, include preservatives, stabilizers, anti-adherents and silica flow conditioners or glidants such as silicon dioxide.

If so-desired, the fixed-dose combination tablets of the present invention may include appropriate amounts of other pharmaceutically acceptable excipients such as vehicles (e.g., lactose, mannitol, potato starch, wheat starch, rice starch, corn starch, and crystalline cellulose), binders (e.g., hydroxypropylmethylcellulose, hydroxypropylcellulose, methylcellulose, and arabic gum), swelling agents (e.g., carboxymethylcellulose and carboxymethylcellulose calcium), lubricants (e.g., stearic acid, calcium stearate, magnesium stearate, talc, calcium hydrogen phosphate, and anhydrous calcium hydrogen phosphate), fluidizers (e.g., hydrous silica, light anhydrous silicic acid), colorants (e.g., red iron oxide), surfactants (e.g., sodium lauryl sulfate, sucrose fatty acid ester), coating agents.

The pharmaceutical compositions of the present invention may further comprise a disintegrant. Disintegrants are agents that aid in the disintegration of the tablets and include, but are not limited to, starch, clays, microcrystalline cellulose, sodium starch glycolate, and cross-linked polymers, preferably, crospovidone. The amount of each excipient can be readily determined by routine experimentation.

The tablets of the present invention may further comprise a coating—a light protective layer that may account for about 0 to about 15% by weight of the tablet composition. The coating layer, which is applied over the entire tablet, may comprise any conventional coating formulations and will include one or more film-formers or binders, such as a hydrophilic polymer like hydroxypropylmethylcellulose, and/or a hydrophobic polymer like ethyl cellulose, cellulose acetate, and one or more plasticizers, such as triethyl citrate, diethyl phthalate, propylene glycol, glycerin, butyl phthalate, castor oil and the like.

The film formers are applied from a solvent system containing one or more solvents including water, alcohols such as ethyl alcohol or isopropyl alcohol, ketones such as acetone, or ethylmethyl ketone, chlorinated hydrocarbons such as methylene chloride, and dichloroethane. Where a color is employed, the color will be applied together with the film former, plasticizer and solvent composition.

Although the present invention has been described hereinabove by way of preferred embodiments thereof, it can be modified without departing from the spirit and nature of the subject invention as defined in the appended claims.

### REFERENCES

1. Conte, U., Maggi, L.: Modulation of the dissolution profiles from Geomatrix multi-layer tablets containing drugs of different solubility. Biomaterials 17, 889-896 (1996).
2. Qiu, Y., Chidambaram, N., Flood, K.: Design and Evaluation of layered diffusional matrices for zero-order sustained-release. J. Contr. Rel. 51, 123-130 (1998).
3. Yang, L., Fassihi, R.: Examination of drug solubility, polymer types, hydrodynamics and loading dose on drug release behavior from triple-layer asymmetric configuration delivery devices. Int. J. Pharm. 155, 219-229 (1997).
4. Abraham, M. A., Shirwaikar, A., Formulation of multi-layered sustained release tablets using insoluble matrix system. Indian J. Pharm. Sci. 59, (6), 312-315 (1997).
5. Nangia, A., Molloy, T., Fahie, B. J., Chopra, S. K.: Novel regulated release system based on geometric configuration. Proc. Int. Contr. Rel. Bioact. Mat. 22, 294-295 (1995).
6. Chidambaram, N., Porter, W., Flood, K., Qiu, Y.: Formulation and characterization of new layered diffusional matrices for zero-order sustained release. J. Contr. Rel. 52, 149-158 (1995).
7. Zerbe, H., Krumme M.: Smartrix system design characteristics and release properties of a novel erosion-controlled oral delivery system, in Modified-Release Drug Delivery Technology, Drugs and the Pharmaceuticla Sciences, vol 126, eds Rathbone M., Hadgraft J., Roberts M, Marcel Dekker, New York—Basel, (2002).
8. Cremer, K., Asmussen, B.: Novel controlled-release tablet with erodable layers. Proc. Int. Contr. Rel. Bioact. Mat. 22, 732-733 (1995).
9. Kim, C. J., Lee, P.: Pharm. Res., 9 (1992)10-16.
10. Langer, R., Peppas, N. A.: Present and future applications of biomaterials in controlled drug delivery systems. Biomater., 2, 201, 1981.
11. Chein, Y. W.: Fundamentals of controlled-release drug administration in “Novel drug delivery systems, fundamentals. developmental concepts, biomedical assessments”, Marcel Dekker, Inc., New York, 1982, Chap. 9.
12. Kost, J., Langer, R.: Hydrogels in Medicine and Pharmacy, N. A. Peppas (Ed), Vol. 3, CRC Press, Boca RAton, Fla., 1987.
13. Gander, B., Gurny, R., Doelker, E., Peppas, N. A.: Pharm. Res., 6 (1989) 578-584.
14. Langer, R.: Polymers for drug delivery and tissue engineering. Ann. Biomed. Eng. (1995) March-April; 23(2): 101-11.

1. A multi-layer oral dosage form, comprising:
(a) a matrix core comprising a therapeutically effective amount of a first drug, wherein the matrix core allows sustained release of the first drug;
(b) a first layer, which is in contact with said matrix core, comprising a first portion of a pharmaceutically effective amount of a second drug, wherein the first layer allows sustained release of the second drug; and
(c) a second layer, which is also in contact with said matrix core, comprising a second portion of the second drug, wherein the second layer allows immediate release of the second drug.

2. A multi-layer oral dosage form, according to claim 1 further comprising in the first layer an additional amount of the first drug, wherein the first layer allows sustained release of the first and second drugs.

3. The multi-layer oral dosage form as defined in claim 1, wherein said matrix core further comprises insoluble polymers and adjuvants.

4. The multi-layer oral dosage form as defined in claim 3, wherein said polymers are selected from the group consisting of insoluble cellulosic materials, polyvinyl acetates, polyvinyl alcohols, polyethylene oxides, metacrylates, and non-crosslinked polyvinylpyrolidone.

5. The multi-layer oral dosage form as defined in claim 3, wherein said adjuvants comprise sugars, colloidal silica, calcium diphosphate, talc and magnesium stearate.

6. The multi-layer oral dosage form as defined in claim 3, wherein said first layer further comprises water-soluble and/or gel forming polymeric materials.

7. The multi-layer oral dosage form as defined in claim 3, wherein said second layer further comprises pharmaceutically acceptable excipients selected from the group consisting of cellulose derivatives, cross-linked polymers, sugars, soluble salts, colorants, fillers, disintegrants, anti-tacking agents and anti-static agents.

8. The multi-layer oral dosage form as defined in claim 6, wherein said first layer comprises from about 15 to about 95% of the second drug.

9. The multi-layer oral dosage form as defined in claim 7, wherein said second layer comprises from about 5 to about 85% of the second drug.

10. The multi-layer oral dosage form as defined in claim 1, wherein said first drug is an NSAID.

11. The multi-layer oral dosage form as defined in claim 10, wherein said NSAID consists essentially of diclofenac.

12. The multi-layer oral dosage form as defined in claim 11, comprising from about 50 to about 150 mg of diclofenac.

13. The multi-layer oral dosage form as defined in claim 12, comprising about 75 mg of diclofenac.

14. The multi-layer oral dosage form as defined in claim 10, wherein said NSAID consists essentially of aspirin.

15. The multi-layer oral dosage form as defined in claim 12, comprising from about 50 to about 150 mg of aspirin.

16. (canceled)

17. The multi-layer oral dosage form as defined in any one of claims 1 to 16, wherein said second drug is an H₂-receptor antagonist.

18. The multi-layer oral dosage form as defined in claim 17, wherein said H₂-receptor antagonist consists essentially of famotidine.

19. The multi-layer oral dosage form as defined in claim 18, comprising from about 20 to about 60 mg of famotidine.

20. (canceled)

21. (canceled)

22. (canceled)

23. (canceled)

24. (canceled)

25. (canceled)

26. (canceled)

27. (canceled)

28. A method for treating and preventing osteoarthritis in patients susceptible to developing NSAID induced gastric and duodenal ulcers comprising administering a multi-layer oral dosage form as defined in claim 1.

29. (canceled)

30. A method for preparing a multi-layer oral dosage form according to claim 2, comprising:
(a) preparing a sustained release matrix core comprising a therapeutically effective amount of a first drug or pharmaceutically acceptable salts thereof;
(b) preparing a sustained release blend comprising a first portion of a pharmaceutically effective amount of a second drug or pharmaceutically acceptable salts thereof;
(c) preparing an immediate release blend comprising a second portion of the second drug or pharmaceutically acceptable salts thereof; and
(d) combining, by compressing, the matrix core of step (a), the sustained release blend of step (b) and the immediate release blend of step (c).

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)
